(19) United States
(12) Patent Application Publication
(10) Pub. No.: US 2024/0000942 A1
(43) Pub. Date: Jan. 4, 2024

(54) Craniofacial implant made of polyether ether ketone (PEEK) with deposits to store and release active substances or active Ingredient

(71) Applicant: Arcosystem SPA, Las Condes (CL)

(72) Inventors: Ilan Bernardo Rosenberg Vaizer, Lo Barnechea (CL); Marcos Alfredo Skarmeta Silva, La Reina (CL)

(21) Appl. No.: 18/158,051

(22) Filed: Jan. 23, 2023

Related U.S. Application Data
(60) Provisional application No. 63/367,226, filed on Jun. 29, 2022.

(30) Foreign Application Priority Data
Jul. 20, 2022 (CL) 202201955

(51) Int. Cl. A61K 47/34; A61M 31/00

(52) U.S. Cl. CPC A61K 47/34; A61M 31/002

(57) ABSTRACT

Craniofacial implant composed of PEEK for the prolonged and sustained release of at least one active ingredient soluble in a pharmaceutically accepted vehicle.

### FIELD OF THE INVENTION

The present invention relates to the biomedicine field. More particularly, it relates to a craniofacial implant made of polyether ether ketone (PEEK) material that has reservoirs to store and dispense substances or active ingredients to the implant site.

### BACKGROUND OF THE INVENTION

In the course of surgical procedures, infections are conditions that frequently develop into emergencies that can determine the failure or success of a procedure. In surgeries where the procedure involves the insertion of a prostheses, periprocedure complications remain a significant concern. One of the most common and complex complication is bacterial surgical infections that can give rise to delayed wound healing, as well as increased monetary costs for the patient, and in severe cases, even death (Chengzhe et al, 2021). These kind of infections account for the 15 to 30% in-hospital infections with a mortality rate of 0.6 to 1.9% (Hernandez et al., 2020).

The incidence of post-surgical infections depends on the site, and ranges from <1 to 2% for joint replacements and up to 10% for spinal surgeries, moreover, infection rates also depend on the health of the patient and are expected to increase among risk patients (aging, diabetic, obese, and those with metabolic syndrome and immunocompromised patients) (Delaney et al., 2019).

In Chile, surgical wound infections have remained as the third most common infection, where its indicators consider specific surgery patients with surgical site wound infections, prosthesis implantation surgeries included among these (MINSAL, 2019).

Several studies have reviewed the postoperative surgical site infection phenomena associated with implantation. In a review study that includes a meta-analysis of 227 articles where 1118 cases of infection were reported, the overall infection rate was 4.87%, this rate being more serious on neurosurgical procedures. In this study, the presence of gram-positive bacteria such as S. aureus, coagulase-negative Staphylococcus and P. acnes were identified (Chen, Y., et al., 2019).

The Kwarcinski J. and colleagues review document highlights a wide variation in reported infection risk for different materials in cranial repair. It is pointed out that these composite materials are to mimic natural bone and assist in restoring function (structurally and aesthetically) to the human but that they are not missing potential infection risks. According to the results of the review, (where a total of 41 articles met the author's inclusion criteria) average infection rates per material ranged between 2.04% and 10.98%. Results indicate that variation exists between materials in regards to total infection risk, however, depending on the compared materials, this value may prove to be be insignificant. Alternative risk factors associated with infection, including operating time, surgical revision and previous infections, have a greater impact on the infection potential than material variation. Comparison of fabrication methods did highlight a notable effect on average infection rate. Trends can be observed showing that materials with greater levels of surface interaction and active support of tissue ingrowth presented greater infection resistance. In the case of PEEK material infection rates varied between 0.00% and 14.29%, with an average infection rate of 7.89%, a standard deviation of 5.16% and a relative risk of 0.75 (Kwarcinski et al., 2017). Inflammation on the surgery site due to the presence of the implant is another difficulty observed.

Prostheses have been continuously updated so that risks associated with infections are reduced, for example, the bone cement has been enhanced to have mechanical and antibacterial properties, so infection risk is reduced. Not so for uncemented prostheses, which currently have limited properties for preventing and treating bacterial infections where available strategies basically involve revision surgery. (Chengzhe et al, 2021).

The use of materials such as PEEK poses as an alternative as material for the manufacturing of implants in clinical applications. This material is biocompatible, chemically stable, and radiolucent and has an elastic modulus similar to that of natural bone (Gu et al., 2021). There are studies that assess the use of prostheses manufactured with PEEK material that exhibit antimicrobial activity after being implanted on patients to reduce post-surgery infection risks.

For example, in 2019, Delaney and colleagues described a PEEK antibiotic reservoir that could be incorporated to a patient as a spinal implant. This implant could achieve slow antibiotic release and/or other compounds, where said reservoir was made with porous material so the controlled and slow antibiotic release was achieved when applying ultrasound (Hernandez et al., 2020).

Studies on prostheses made with PEEK materials coated with nanoparticles or in alloys with other materials that could present antibacterial activity have been carried out. The 2018 Yan and colleagues document describes a silver nanoparticle and gentamicin sulfate coating constructed upon a porous PEEK surface (Yan et al., 2018). The coating greatly enhanced the bactericidal efficiency to Gram-positive bacteria and Gram-negative bacteria, moreover, while the number of viable bacteria adhered to this coated PEEK was lower than that adhered to uncoated PEEK sample (Yan et al., 2018).

Xue and colleagues developed in 2020 a layer-by-layer (LBL) deposition method with controlled cycles to rapidly construct brushite (CaHPO4·2H2O) (CaP) layers containing gentamicin sulfate (GS) on PEEK to obtain CaP-and-GS modified PEEK (PEEK/CaP-GS) (Xue et al., 2020). Authors describe that in vitro antibacterial experiments illustrated that all of the PEEK/CaP-GS samples had excellent and sustained antibacterial properties, while cell proliferation experiments revealed the acceptable biocompatibility and cell osteogenic differentiation (Xue et al., 2020). On the other hand, on the 2016 Lazar and colleagues document a fiber-reinforced composite coated with gentamicin implant for craniofacial reconstruction with antimicrobial properties was developed. Results on this study showed that bacteria were efficiently inactivated in direct contact with gentamicin coatings (Lazar et al., 2016).

Chengzhe and colleagues, on their document published in 2021, present an study where a PEEK implant was developed to simultaneously fight off bacterial contamination and promote osseointegration by sustained release of moxifloxacin hydrochloride (MOX) and osteogenic growth peptide (OGP) from polydopamine (PDA)-coated porous sulfonated PEEK (SPK) surface. (Chengzhe et al, 2021). Results indicate that the MOX/OGP PDA-modified SPK (SPD-MOX/OGP) surface exhibited a durable and excellent antibacterial effect against planktonic/adherent Staphylococcus aureus and Escherichia coli in vitro. Besides, a emarkable enhancement in specific cell adhesion, proliferation and osteogenicity were observed on the SPD-MOX/OGP substrate due to the presence of OGP and PDA molecules compared to all other groups. (Chengzhe et al, 2021).

Implants for bacterial control on the implant site have also been described on patent documents. For example, the CN101432030B document describes a a three-dimensional body or reservoir including one or more implantable substances. Said reservoir can receive amounts of liquid. The reservoir and the liquid can forma conformable implantable material such as a putty. Other document such as WO2007001624A2 divulges an implantable medical device for use in the treatment of osteonecrosis. Said implantable device is adapted for insertion into one or more channels or voids in bone tissue; a plurality of discrete reservoirs located in the surface of the at least one implant device body; and at least one release system disposed in one or more of the plurality of reservoirs, wherein the release system includes at least one drug selected from the group consisting of bone growth promoters, angiogenesis promoters, analgesics, anesthetics, antibiotics, and combinations thereof.

The US20170056565A1 divulges a biocompatible clip made of PEEK for the treatment of surgical site infections, that is attached to an implant. This clip comprises a reservoir and a reservoir opening, wherein the reservoir opening is sealed with a pressure responsive material that can be ruptured upon application of a force to said material. Said reservoir is filled with a therapeutic selected from the group consisting of: an antibiotic, anti-viral, pain medication, growth factor, anti-fungal, antimycobacteria chemotherapeutic, osteogenic, non-toxic factor or metabolite, such as ascorbate to increase bone formation, parathyroid hormone, peptides, peptoids, NSAIDs, analgesics, or combinations thereof.

The U.S. Pat. No. 8,821,912B2 document describes methods of fabricating implantable medical devices, than can be manufactured with PEEK, having antimicrobial properties. The antimicrobial effect of these devices is produced by incorporating ceramic particles containing antimicrobial metal cations into molten PEEK resin, which is subsequently allowed to cool and set in its final shape achieved by injection molding, cutting and machining or other techniques.

Although advances on the fabrication of prostheses with antibacterial properties have been made and described on the state of the art, there is still a need to avoid the onset of infections on the implant site on a prolonged and controlled manner.

It is acknowledged on clinical practice that the first 48 to 72 hours after implant insertion surgery are vital, so implant alternatives are required to be able to sustain a prolonged antimicrobial environment during this time course to avoid infection onset.

Current proposals until now refer to implants with close-contact momentary antimicrobial action, or that require the functionalization with nanoparticles, making the implant production technically complicated and more expensive. In other cases, clips or external devices are attached to the implant surface, this involves the complication of the production and spatial adjustment of the complex cranial implant on the patient.

Therefore, there is a need for a craniofacial implant that can be able to release clinically relevant active ingredients of interest on a constant and prolonged manner into the implant site. This would allow on some cases, to exert or promote a prolonged and controlled antimicrobial environment during the first hours after the implant fixation to avoid the onset of infections, and that also is a simple implant with a lower production and working cost.

The present application describes an implant, particularly a craniofacial implant made from PEEK, that comprises one or more storing reservoirs or chambers that can hold active ingredients or substances in liquid form or liquid format therein that can be released in a gravity-controlled and gradual manner on the bone replacement site.

### DETAILED DESCRIPTION OF THE INVENTION

The invention relates to a craniofacial implant made of polyether ether ketone (PEEK) that has reservoirs to store and dispense or active subtances or ingredients, because said implant comprises:

One or more internal ovoid chambers or reservoirs (1) with walls 2.5 to 4 mm thick, that store a liquid or active substance in liquid form to be released in a gradual and prolonged manner into the implant site,
tubes (2) that interconnect the internal chambers or reservoirs with the entry and exit openings, defined as:
   a) an entry tube that connects the entry opening (3) with the reservoir (1) in such a way that it includes a funnel system that includes an 1.5 to 2.0 mm diameter liquid entry or input opening that widens to a 2.0 to 3.0 mm wide tube that then continues to the reservoir.
   b) an exit tube that connects the chamber or reservoir (1) with the exit opening (4) that includes a transition funnel system that is 0.7 to 2.0 mm wide on its upper part and 0.7 to 1.5 mm wide on its lower part, depending on the diameter of the exit opening (4),

One or more entry openings (3) on the upper part of the implant depending on the number of reservoirs, where said opening is the entry or input for the liquid to reach the reservoir or reservoirs (1), where the opening is 1.5 to 2.0 mm wide and is blocked by a PEEK filament plug or a titanium screw,

One or more exit openings (4) on the lower part of the implant depending on the number of reservoirs as gradual and prolonged release of the liquid or active ingredient to the implant site, where the exit opening is 0.7-1.5 mm wide and has a plug or system to seal the implant to prevent the escape of the liquid when the implant has not been properly set yet.

The craniofacial implant described on the present invention avoids post-implant infections, particularly, bacterial infections. This effect is achieved thanks to the sustained, controlled and prolonged release of a liquid, active substance or ingredient in liquid form.

The inventors have specifically defined the design and spatial location of each component inside the implant so that big enough chambers or reservoirs are available to accumulate a considerable amount of liquid or active substance or active ingredient in liquid form, but that at the same time the implant does not lose its structural characteristics and properties.

The inventors carried out a series of experimental iterations to define the best structural conditions for size, length and diameter of each part or component of the craniofacial implant of the present invention. It posed a technical and design challenge to produce an implant with one or more chambers or reservoirs system, or a rather a multi-reservoir inner liquid storage with appropriate capacity. More complex an important, how the liquid of interest is added and released from the implant to the patient.

So, it was empirically defined on the iterations that the reservoir may contain one or more storage chambers or reservoirs depending on the stored liquid release requirement. Particularly, implants with a mL filling capacity reservoir and implants with 2 or more reservoirs to contain at least 2.5 mL of the active substance or ingredient in liquid format or format were defined.

Even when it was initially observed that controlling the liquid flow and the entry and exit openings to achieve its controlled and sustained release was complex in the implants with two or more chambers, the inventors included on their experimental iterations the definition of each entry and exit opening diameter, along with the length and spatial disposition of the sealing components (plug) that prevent the release of the liquid when the implant has not been used yet and that allow for its proper performance.

When the inventors assessed the diameters of the entry and exit openings, they also encountered different problem areas for the proper and sustained release of the liquid or active substance that is stored within the reservoirs. In the present invention the conditions for the reservoir or capsule and entry and exit opening sizes correlate with the regulation of exit or release for the active liquid or active substance in liquid form from the reservoirs to the its environment.

As part of the experimental iterations, the inventors defined that the entry openings on the upper part of the implant to load the liquid or active substance, must be of an appropriate diameter so it can be coupled with a syringe that allows the entry of said liquid or active ingredient to the implant. In turn, the orifice must not collapse nor allow for the liquid leakage or overflow. In that sense, the inventors have defined that the entry tube that connects the entry opening (3) with the reservoir (1) includes a funnel system that includes an 1.5 to 2.0 mm diameter for liquid entry or input that widens to a 2.0 to 3.0 mm wide tube that then reaches the reservoir through said tube, where the entry orifice is 1.5 to 2.0 mm wide.

As part of the improvements, an additional section was added to the entry orifice of the implant to insert a piece of filament for it to work as a plug. This plug can be manufactured from PEEK material or can consist on a titanium screw.

In the case of the exit openings on the lower part of the implant as an opening for the gradual and prolonged output of the liquid or active substance at the implant site, the inventors observed on the tests that were performed in the application examples that there was a need for further regulation of the liquid exit from the reservoirs or capsules so the effective release and exposition time on the environment or implant site is enhanced. It is of importance to note that the inventors have defined all of the conditions of the funnel system, entry and exit diameters and minimal length and width for each structure within the implant design, in such a way that it allows for the prolonged release os the active substance in liquid format.

This way, the inventors defined that the implant must have one or more exit openings (4) on the lower part of the implant depending on the number of reservoirs as gradual and prolonged release of the liquid or active ingredient to the implant site, where the exit opening is to 1.5 mm wide and has a plug or system to seal the implant to prevent the escape of the liquid when the implant has not been properly set yet. Regulation of the size and diameter of the exit opening and the remaining measurements that the inventors have defined for each structure within the implant allow for the gradual release of the liquid or active substance in liquid format.

In the case of the tubes that interconnect to the chambers or reservoirs, they are arranged as tubes in the upper part of the chambers or reservoirs that become the entry orifices for the incorporation of said liquid to said reservoirs and as tubes that go from the deposits to the lower or bottom part of the implant and that become the exit openings with controlled diameters and defined at is has already been described on this document. The tubes for all the systems, both for the entry and the exit system must be at least 10 mm long and no more 15 mm long, this minimum length results from the tests that were developed for the liquid to enter in an appropriate way to the chamber or deposit.

In one of the forms of the invention, the release of the liquid or active substance in liquid form is directly related with the delivery of active ingredients to the implant site after its inclusion on the body of the patient. In that sense, the release of active substances in liquid format from the implant to the implant site allows for an specific effect to take plane in a sustained manner at the implant site after the surgical procedure. In one of the forms of the invention, the prolonged and sustained release from the first days after the implant allow to avoid or decrease postoperative infections. The sustained release of active substances during the first hours after a surgical procedure, such as antibiotic and anti-inflammatory substances, helps to avoid bacteria colonization that may cause infections at the implant site. As it is shown in the application examples, particularly FIG. 1, as part of application example 1, where the implant drip releases liquid. The inventors defined specific sizes for said drip release as part of a prolonged release mechanism on the implant zone.

In one of the forms of the inventions, the release of active substances or ingredients in liquid form from the implant allow for a local clinical effect depending on the active substance type, without being limited to, antibiotics, anti-inflammatories, analgesics or other active substance. Chemotherapeutic agents or any other soluble active substances that may constitute a solution are also included as active substances. As part of the reach of the invention, at least one active substance may be included, with the possibility of including more than one, where said substances may be included within the same reservoir or on separate reservoirs.

The inventors have shown in the application examples 2 and 3 that the implants that are within the scope of the invention can release a liquid or active ingredient in liquid form in a prolonged manner from one or more chambers or reservoirs or capsules included within the inner structure of the implant. The physical, spatial and sizes arrangements of each implant component allow for the release of the liquid substance fro the 24 hours and up to 11 days after the incorporation of any substance in that format. Even in simulated movement conditions (head movement simulation of the patient) this sustained and prolonged release was observed from the implant to its environment for several days.

These results show that the implant that is described in this invention is functional and releases in a sustained and prolonged manner the liquid or substance in liquid form included within the chamber or chambers of the implant.

It is important to emphasize that the results that were shown in the application examples 1 to 6, in particular for the 5 and 6 examples, while it is true that the tests were performed using vancomycin as active substance, both the tests and results are valid and can be extrapolated for ant other active substance that can be solubilized in any appropriate solvent, in this case pharmaceutically acceptable solvents or vehicles. An example of solvent can be water, physiological saline solution, potassium dibasic phosphate buffer, saline phosphate buffer or any other acceptable buffer or vehicle for its administration on a living being, particularly a human being. In that sense, it falls within the scope of this invention the possibility to include any active substance that can be soluble in a pharmaceutically acceptable vehicle in accordance to what has been previously stated.

In example 5 the analysis for the release concentration and rate is shown. The amount of released vancomycin in the first hour was of 13087.5 μg. Then, in hour 2, a decrease in the released amounts can be observed with 548.5 μg of active substance that then is increased at hour 4 with 1232.5 μg of active substance. For the 8 and 12 hours of the assay an increase of 3505.5 μg and 4309.4 μg was also observed, respectively.

Regarding the release rate at the first hour a high vancomycin release rate was observed with a mean value of 218 μg/mL, that then decreased in the time elapsed from the first to the second hour, to then increase and maintain the prolonged release. This release kinetic allows to indicate that there is an initial fast or higher release of the active substance to then keep an steady and prolonged release.

In application example 6 it is demonstrated that the active substance loaded within the reservoir or reservoirs remains stable and does not lose its structure nor is degraded, so it remains functional. In this particular case, the stability of a vancomycin solution was assessed in an implant for 24 hours, showing no indication of significative degradation of the active substance, so the reservoir does not affect the stability of the to be released active substance.

This information allows to report that the active substance is properly released with a release kinetic appropriate for the release of active substances or pharmaceuticals in the craniofacial implant zone, where an initial fast release to achieve a therapeutic effect is observed, then a decrease and then a steady release are observed for at least 43-48 hours. Furthermore, storage of the active substance or a solution of it within the reservoir does not affect its stability.

The implant described on this invention refers to a craniofacial implant manufactured by 3D printing with customized PEEK material. Said implant comprises two openings or orifices, one at the top and the other at the bottom, that allow for the fill and exit of the substances to be released, respectively, as it has been described for the present invention.

In the implementations of the invention, the containers or deposits or chambers or reservoirs described in the implant, can incorporate substances or medicines or active substances in liquid format or form, which can be released in the site where the bone replacement procedure was performed.

In the implementations of the invention, the implants with deposits or chambers or reservoirs particularly include medicines or actives substances like antibiotics and/or anti-inflammatories and/or analgesics in order to achieve success on the incorporation of said implant. It also falls within the scope of this invention for the drug or active substance to correspond to a chemotherapeutic agent or any other substance that may be dissolved and obtained as a liquid solution. It is also important to point out that because the implant may have more than one reservoir, it also falls within the scope of the invention

The release of substances through the reservoir, chamber or deposit of the customized implant made from PEEK, described on the present invention is a result of using the action of gravity, that allows for a gradual release of said compounds. The release depends on the diameter of the exit opening and size of the other structures of the implant.

Among the implementations of the invention, the development of the customized implant includes inner chambers or reservoirs and an interconnected tube system, where said reservoirs can store liquid substances that can be released due to gravity force. Particularly, an implant with upper chambers or reservoirs that allows for the substance to flow using gravity is presented, where said substances can be antibiotics and/or anti-inflammatories and/or analgesics.

In implementations of the invention, it is described that the liquid substances can be incorporated into the inner reservoirs of the implant via the use of a surgical needle.

### Definitions

The present document describes a craniofacial implant, wherein said implant refers to any implant that may be placed in cranial and/or face area in a patient.

The craniofacial implant described on the present invention allows for the prolonged and sustained release of active substances or ingredients, particularly those on liquid form, to allow for a specific therapeutic effect on the implant site. Several types of substances or active ingredients are included in pursuit of analgesic, anti-inflammatory or antibiotic effects, chemotherapeutic agents or any active substance that is able to be included in a solution. One of the forms of the invention allows to avoid post-implant infections, particularly, bacterial infections. This effect is measured by a decrease of the bacterial count. In that sense, when a reference is made to the decrease of the bacterial count, a reference is being made to the decrease on the UFC or UFC/mL bacterial count.

When a customized PEEK is being referenced in this document, it is stated that the implant described on the invention is being manufactured from PEEK and is personalized according to the characteristics of the implant that the patient is going to receive.

When the “receptacle”, “chamber”, “deposit”, “reservoir” and/or “internal chamber” and/or “capsule” term is being referenced in this document it is stating a cavity that contains or could contain a substance on its interior.

The “postoperative” term refers to the period that follows after a surgical procedure and that finishes with the rehabilitation of the patient. The “perioperative” term corresponds to the surgical procedure time period, which ranges from the preparation of the patient until their recovery.

When in the document “active liquid”, “active substance” or “active ingredient” is being indicated, it is making reference to every substance or mixture of substances that have a function and/or particular activity in the composition of a medicinal product.

In the document the term “iterations” is indicated, this term makes reference to a repetition of steps for a particular purpose. On this invention particular case, the iterations make reference to the repetition of the implant manufacturing and performance of tests on it to obtain an implant with the required functionality.

### FIGURE DESCRIPTION

FIG. 1.—Image of the iteration 1 prototype for implant development. The prototype 1 composed of multiple-deposit reservoirs is presented. Drip failure and liquid leak from the reservoir. A) shows the structure of the implant prototype; B) describes the prototype parts, where (1) refer to reservoirs containing liquid or active ingredients with a total capacity of 3 mL, (3) liquid or active ingredient input via a 1.25 and 1.5 mm wide syringe; and (4) 0.5, 0.7 and 1 mm wide exit openings for the liquid or active substance.

FIG. 2.—Image of the iteration 2 prototype for implant development. In A) the single-chamber or reservoir Prototype 2 implant is presented (1). B) describes the entry of the liquid or active ingredient through a 26 mm long tube (2) and the 4.7 mm thick prototype is shown with a thick black arrow. Prototype 2 failed because the implant loses mechanical capabilities, because of the weakening of the internal implant structure.

FIG. 3.—Image of the iteration 3 prototype for implant development. Prototype 3 implant with 2 independent reservoirs for active liquid or ingredient deposit. The prototype failed because it showed issues with the exit tube diameter in one of the reservoirs. In (1) the liquid or active substance reservoirs are shown; (3) liquid or active substance entry openings via a syringe; (4) liquid or active substance exit openings.

FIG. 4.—Image of the iteration 4 prototype for implant development. Material is added to the inner part to achieve at least a 2.5 mm thickness on each side of the chamber or reservoir (1). In (2) 1.4 mm diameter tubing, (3) the entry opening for a 1.5 mm diameter syringe, (4) 0.75 mm or 0.5 mm diameter exit opening.

FIG. 5.—Image of the iteration 5 prototype for implant development. An implant with 2-inner chambers or reservoirs with 3.5 thick separation pairs and with defined liquid entry and exit openings is provided. A) digital diagram of the implant, b) implant mold.

FIG. 6.—Image of the iteration 6 prototype for implant improvement: plug on the tube of the liquid entry opening to the implant and funnel-like system. In A) an image of the upper part of the implant is presented, where the entry tube and the entry opening can be appreciated, where there a a PEEK scrap or piece is in place to work as a plug that prevents the escape or reverse flow of the liquid contained within the implant to the entry opening. In B) an implant that incorporates improvements such as a funnel-like system for the liquid entry is shown, this system may take a variety of diameters for each tube according to those shown in the image with a 1.5 mm wide opening for the syringe needle that provides the liquid for the implant that widens to a 3.0 mm diameter tube towards the chamber or reservoir. In said implant, the entry opening exhibits a 2.0 mm diameter exit opening tube with a funnel-like system that then narrows its diameter to 1.3 mm ate the exit opening. The implant shown in the image has a single 5 mL reservoir with 2.5 mm wide wall thickness. In 6C) presents the plug or sealing system for the implant in the exit opening to prevent the liquid from escaping when not in use (see 6C figure).

FIG. 7.—Image of the iteration 6 prototype for implant improvement: plug on the tube of the liquid entry opening to the implant and exit funnel-like system. In A) the implant prototype with 52.2 mm wide and 52.86 mm tall dimensions for the reservoir is presented. B) presents an implant prototype with two reservoirs or chambers that comprises a 24.43 mm wide and 46.7 mm tall left reservoir and a 24.47 wide and 46.84 mm tall right reservoir.

FIG. 8.—Photograph of the fluid release test from an implant of the present invention (application example 2). Different shades of blue are observed consistent with the release of water-diluted methylene blue when the implant is laid out in a container. Release timing was valued from left to right at 1, 2, 3, 4 and 6 hours.

FIG. 9.—Photograph of the emptying time verification of an implant reservoir (application example 3). In A) a photograph of the obtained results in the observation of the samples obtained from the 24, 72, 96, 120 and 144 hour time frames is presented. From left to right, at 24 hours little coloration of the environment or liquid release from the implant is observed. At the intermediate timeframes, that is, 72, 96 and 120 hours, an increase release of the liquid is observed, showing a light blue shade coloration of the environment (container) and then at 144 hours the shade changes to a strong light blue shade. At the 168 hour evaluation a greater release of the contents inside the reservoir or capsule of the implant into the environment (container). In B) a photograph of the exit or outlet opening for the liquid contained within the implant is exhibited, in this case the liquid being water-diluted methylene blue. The release of the liquid is shown from the exit opening with a stronger shade of blue.

FIG. 10.—Image of the implant with reservoirs and tubes for the gradual release of an liquid or active substance to the brain. In A and B the image of the implant integrated into the skull bone of the patient is presented. In A, the detailed inner part of the implant is observed. In B, the implant is seen from the inner part of the skull, where the exit openings of the reservoirs release the liquid towards its inner part and the brain.

FIG. 11.—Graph for released vancomycin from the implant (μg) at the evaluated time periods. A representative graph for this release kinetic is shown, considering released μg for the 0, 2, 4, 8 and 12-hour sampling periods.

FIG. 12.—Vancomycin release rate from the implant per sampling period. A graph for the release rate in μg/min in accordance with the evaluated 0, 2, 4, 8 and 12-hour sampling periods.

### APPLICATION EXAMPLES

### Example 1: Development of Prototypes for Implants with Reservoirs and Tubes for the Controlled Release of Liquids or Active Substances

The development of the implant prototype is part of the present application example, considering the iterations and corresponding experimental analyses to obtain an implant that consists on inner chambers or reservoirs interconnected with tubes in such a way that a liquid or active substance can be stored in it, but that also allow for its prolonged release during the first 24-48 hours after the incorporation of the implant to the patient.

A previously designed 3D implant was used for testing, where one or more inner storing chambers or reservoirs were added to it.
First test iteration: Implant with more than one chamber or reservoir system

The first implant prototype that was developed had several inner chambers or reservoirs and an interconnected tube system, so each reservoir could store liquid within (the test was made with antibiotics) and that liquid could move due to gravity from the chambers that had more liquid to those that had less.

This prototype has three liquid inlets through a 0.8 mm; 1.25 mm and 1.5 mm wide straight tube. In that sense, the inventors observed that the diameter of the entry opening is crucial because it must be big enough to allow for the proper loading of the liquid with a syringe to the inside of the implant, but at the same time it must not be too big to allow for the liquid to overflow and release during the loading process. According to the tests that were done on this prototype, the best diameter was 1.5 mm.

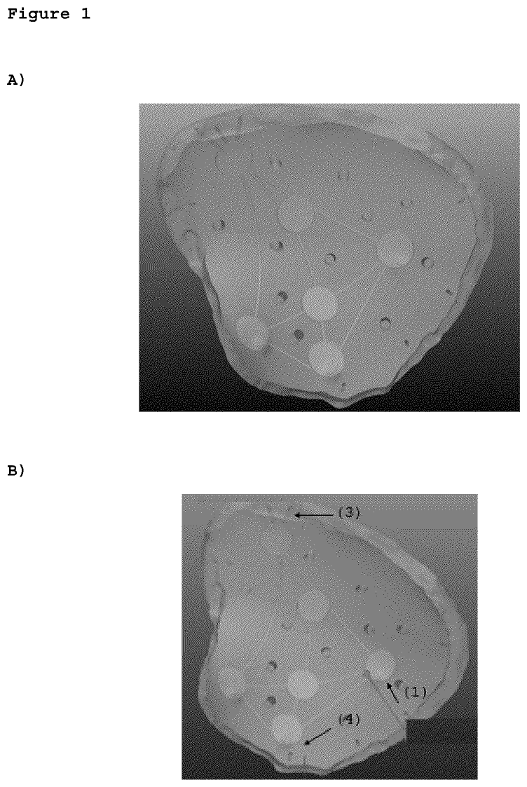

This first prototype worked in regard of the liquid exit or release because several liquid drip exits were observed, but because the tube system was so extensive and with small diameters some of these exits did not work. The tested diameters for the exits were 0.5 mm, 0.7 mm and 1 mm. In this case, when the implant did not completely appropriately work, it was observed that the 0.7 mm exit opening allows for a controlled dripping liquid release. The 0.5 mm exit was blocked and the 1 mm exist exhibit a very high output flow (FIGS. 1A and 1B) In table 1, the results for the liquid entry and exit openings diameter definitions to be included in the implant so it can be released on the implant insertion site are defined.

TABLE 1
Results for the implant entry and exit openings determination.

| Implant entry opening | | Implant exit opening | |
|---|---|---|---|
| Tested | | Tested | |
| diameter | | diameter | |
| (mm) | Result | (mm) | Result |
| 0.8 | Does not fit | 0.5 | Opening is clogged |
| | | | and liquid or active |
| | | | substance is not released |
| 1.25 | Does not fit | 0.7 | Controlled sustained |
| | | | dripping realease of |
| | | | the liquid |
| 1.5 | Correct liquid | 1 | High exit flow rate with |
| | entry | | quick release of the |
| | | | liquid or active substance |

Second Test Iteration: Implant with One Chamber or Reservoir

A previously designed 3D implant was used for testing, where one or more inner liquid storing chambers or reservoirs were added to it.

In first place, the tube system and the connection or interaction type with the chamber or reservoir was designed. A liquid inlet that moves towards the reservoir was provided, as well as an outlet that it above it, this outlet has a pyramidal shape so the speed and amount of deposited liquid can be controlled.

Then, another design challenge was to define the total inner capacity of the chamber or reservoir.

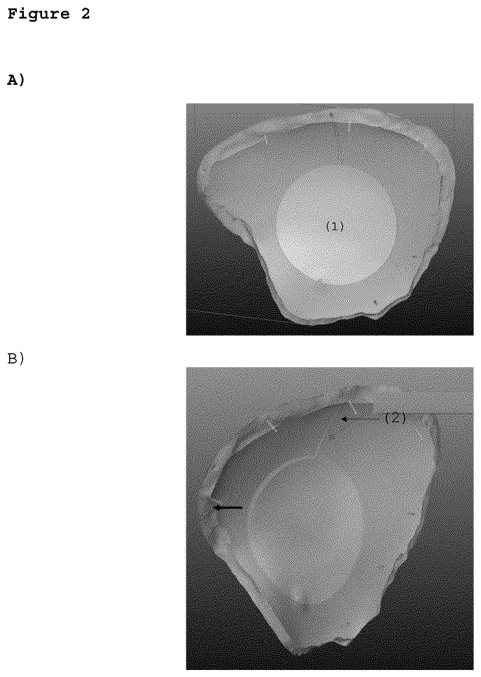

The first test was performed with an implant prototype that included a reservoir with an inner capacity of 2 mL. An 85.62 mm×99.7 mm, 4.7 mm thick implant was provided, comprised of a sole chamber or reservoir with a 26 mm long tube connected to it (see FIGS. 2A and 2B)

To assess the implant performance, in particular the release of a liquid or active substance, a test was performed by adding liquid water to the implant using a syringe. It was observed that the single 2 mL capacity chamber is too big because the implant loses mechanical capacities due to the weakening of the inner structure of the implant.

To solve this problem and to allow for the implant to show proper mechanical properties, it was stated that the walls of each chamber should be at least 2.5 mm thick. When implants that incorporated these considerations were tested, theses implants kept their mechanical properties.

Third Test Iteration: Improvement of the Implant with More than One Chamber or Reservoir System

A second implant prototype or improvement was performed with two independent chambers or reservoirs, each one of them with a liquid inlet and exit. Each chamber has a total capacity of at least 2 mL, so the total amount of liquid capacity considering both chambers may reach up to 5 mL.

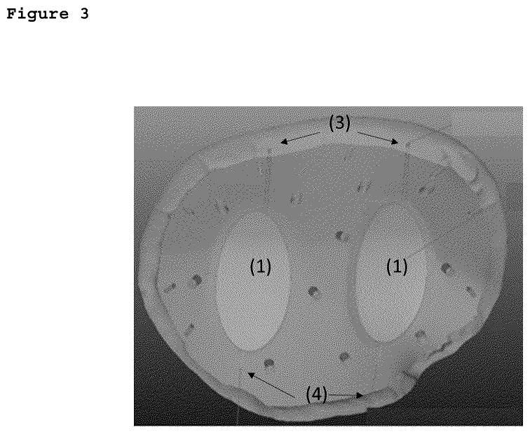

The tested implant is approximately 20% bigger that the previous one, so a bigger area is needed to allow for bigger storing liquid capacity without decreasing the mechanical properties of the implant. The implant comprises a tube that is connected to each reservoir, where said tube has a syringe-like 1.2 mm diameter inlet opening. The implant also has a 0.5 mm diameter exit tube for one reservoir and a 0.35 mm diameter exit tube for the other reservoir (FIG. 3).

In this case the test proved partially successful because only one of the chambers meets the objective of letting the liquid flow in a dripping manner, the other inlet is completely blocked due to its small diameter because of the manufacturing process.

Fourth Test Iteration

The third iteration is based on the same prototype than the last one, but with a big difference, material is added to the inner part so the walls on each side of the chamber or deposit is at least 2.5 mm thick. This, in accordance with what was observed on the second iteration test improves the mechanical conditions and properties of the implant.

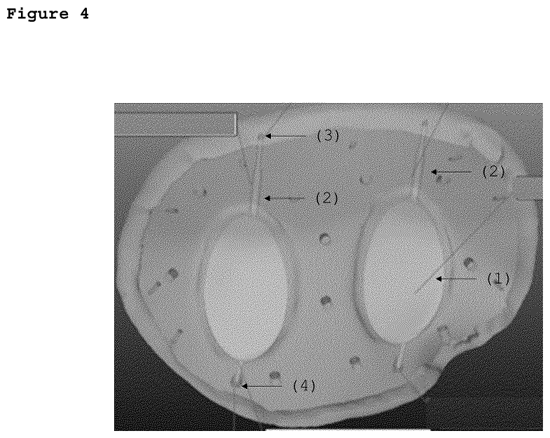

As part of this prototype, the liquid exit tube diameter was increased, but to meet the requirements for controlled liquid release, a funnel system was included on its upper part (FIG. 4).

This implant has two different exit diameters, 0.75 mm and 0.5 mm. It was observed that the best exit diameter was 0.75 mm. In the case of the 0.5 mm diameter exit the liquid does not flow in a simple manner, so it proves difficult to control liquid release.

When material was added to the reservoir area, the intended effect of the implant keeping its mechanical capabilities, while showing excellent storing capacity in both chambers without affecting its quality and integrity was met.

Fifth Test Iteration

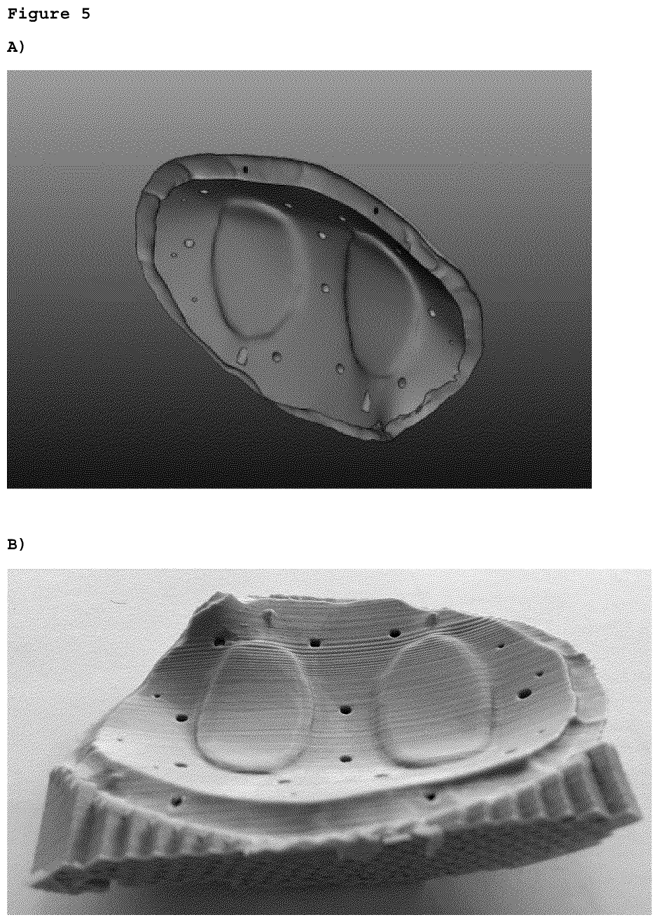

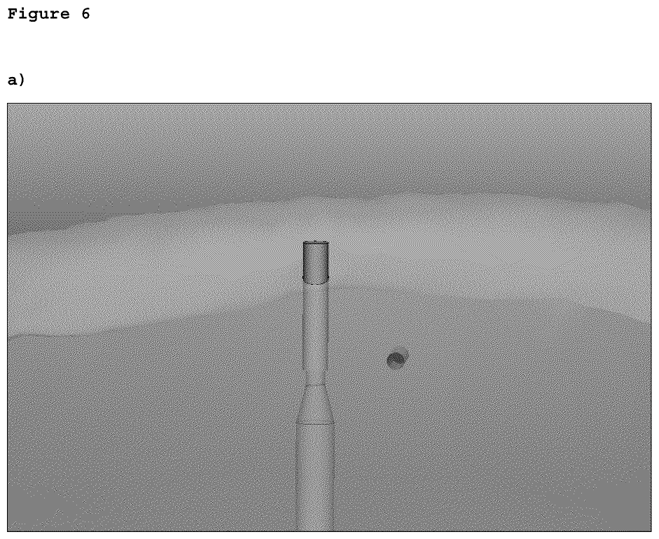

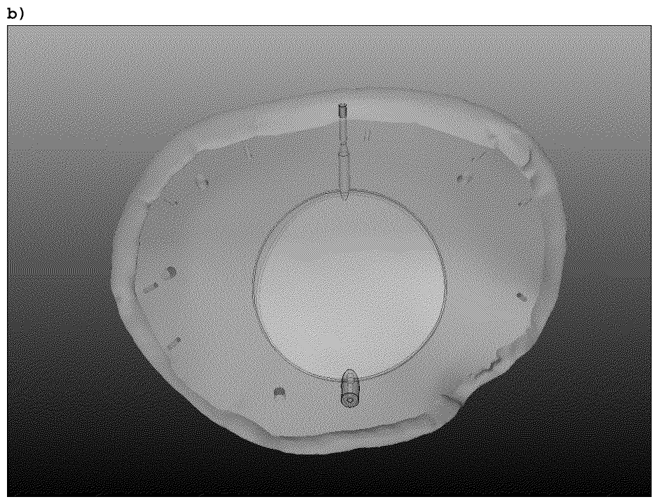

Afterwards, in a subsequent iteration for the previous prototype, storing chambers were arranged on the inner part of the implant while keeping a thickness at least 3.5 mm between the reservoir and the outer part of the implant. To obtain the same thickness on the inner part of the implant a chamber thickening process was carried out to obtain a thickness of at least 3.5 mm, this test proved to be a great success (FIG. 5).

Therefore, the implant can be comprised of one storing reservoir gives its wall thickness is 2.5-4 mm. preferably 3.5 mm.

Sixth Test Iteration: Final Definitions for the Liquid Input and Exit Systems

Considering each of the problems that were observed on previous iterations respecting the control for liquid input and implant site release, several improvements were performed on the prototype implant.

Plug in the Input Opening for the Liquid to be Stored in the Implant

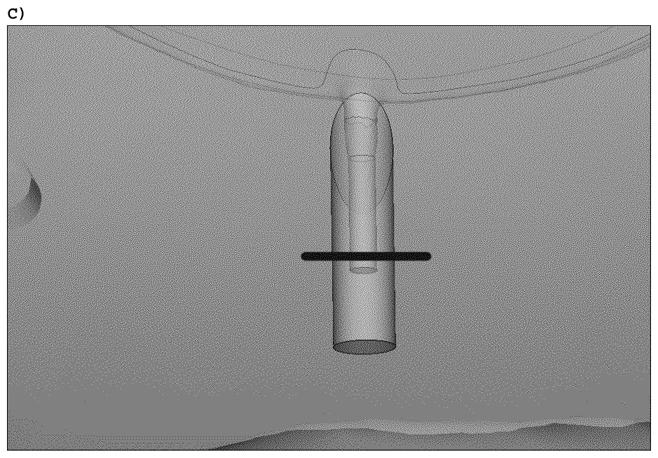

A piece or scrap made with the same material that the implant is constructed of (PEEK) was added at the staring section of the tube, where the liquid enters the implant, so it can work as a plug that prevents liquid stored within the implant from escaping and overflowing from the inlet orifice. The filament that was used has a 1.75 mm diameter, but thermoplastics can dilate, so the starting section of the filament is 0.25 mm thicker than the filament itself. Therefore, the adequate diameter to block the exit with this filament is 2.0 mm (see FIG. 6A).

When the implant is installed, this scrap or filament can be easily disposed of (removed, taken of, pushed out) and once the implant has taken place on its proper site, this opening is covered by the bone on the implant site.

In this case it is also technically possible to include an 1.5 mm titanium screw that is placed in at the exit to work as a plug or seal.

Funnel System in the Tube at the Liquid Inlet Opening

A funnel-like system was incorporated at the liquid inlet opening, this system varies the diameter of each tube, and is designed to achieve that the liquid at the interior of the implant does not overflow to the opening. It is a second method to prevent liquid from escaping the tube system through the inlet opening. The funnel system implies that the inlet tube must have a 1.5 mm diameter for the input of a syringe that loads the liquid to the inside of the implant and then widens to a 3.0 mm tube. In other words, a funnel point or transition exists to avoid the liquid of leaking back to the opening (see FIG. 6B).

Output System in the Exit Tube for the Liquid Release to the Implant Site

The output system was improved by defining the liquid exit opening diameter to be from 0.7 mm to 1.5 mm depending on the geometry of the implant. In this new prototype, the final tube for liquid release incorporates a funnel system whose objective is to facilitate the emptying of the chamber to be more controlled and prolonged (see FIG. 6B).

Furthermore, in this test plug or seal system was added to the implant to prevent liquid from escaping (see FIG. 6C), once the implant in on the operating room, the surgical staff must cut a part of the cylinder where the exit orifice will be exposed so the liquid may be released onto the body.

General Measurements of Implants and Reservoirs

Regarding to overall dimensions, the implant on its full extension will depend on the implant site and each patient. For the chamber or reservoir sizes some general dimensions and sizes were defined.

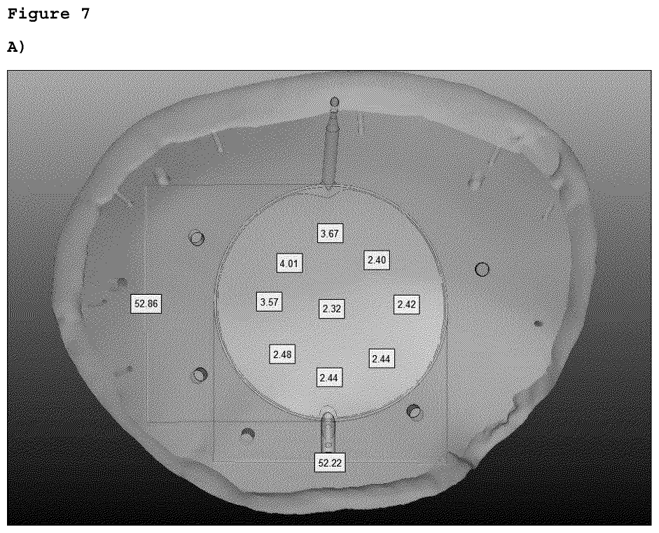

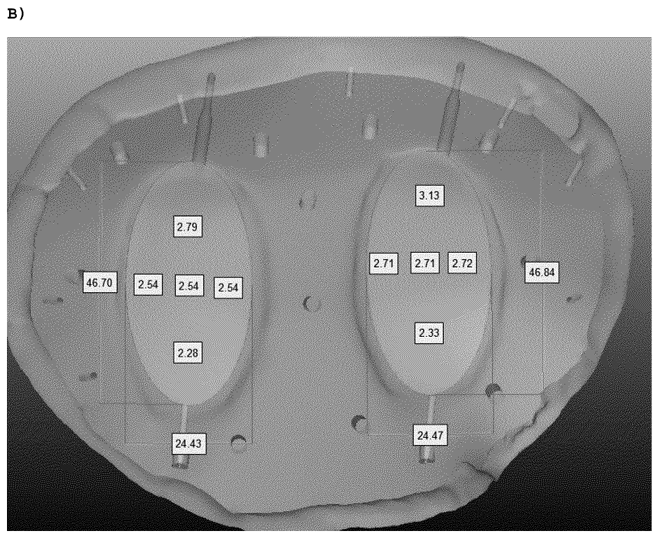

In the prototype implant with one chamber or reservoir (to be used as application example 3) dimensions were 52.2 mm wide and 52.86 mm long for the reservoir (FIG. 7A).

The assessed implant prototype with two reservoirs or chambers comprised a 24.43 mm wide and 46.7 mm tall left reservoir and a 24.47 wide and 46.84 mm tall right (7B) reservoir.

The tubes for all the systems, both the entry and the exit systems, must be at least 10 mm long and no more than 15 mm long, this minimum length is due the tests that were developed for the liquid to enter in an appropriate way to the chamber or deposit.

### Example 2: Photograph of the Fluid Release Test from an Implant of the Present Invention

With the objective of estimating the emptying time of the chamber or reservoir an implant was produced via 3D printing, that is composed of an inlet 2.0 mm filament seal to block the leaking of liquid, an entry tube with a funnel system with an 1.5 mm diameter syringe input and a subsequent exit diameter of 3.0 mm. The tested implant has a 5 mL capacity chamber or reservoir with 2.5 mm thick walls. The outlet tube has an exit orifice with a funnel or controlled release system with a 2.0 mm diameter tube that transitions to a 1.3 mm outlet (FIG. 6 b).

In this experience, the implant was filled with 5.1 mL of liquid on its interior, where said test liquid was a solution 4 parts water and one part methylene blue.

The protocol consisted on injecting the 5.1 mL of solution on the already defined implant inlet and then submerge the implant on a container with water, preserving the geometrical disposition as it would be installed as a prostheses. Then the implant was left in that container for an undetermined time period, corroborating at every hour if the water had changed with the colorant. For testing conditions, the test was performed on static conditions, that is to say, without implant movement, or, or on dynamic conditions simulating the head movement of the patient. This last test allows to check if this condition affects the liquid release rate from the interior of the implant.

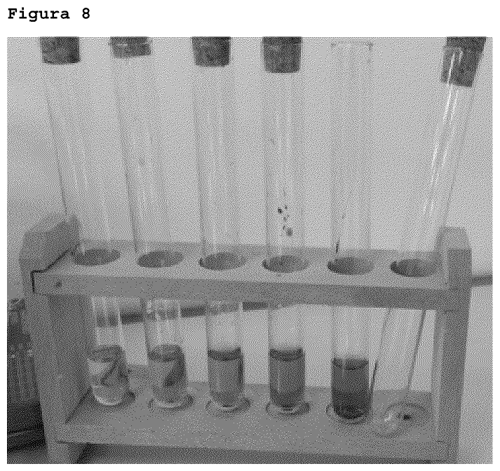

It was observed that during the first hour the liquid that was collected from the container was almost transparent with a very light blue hue or coloration. Increase in the blue shade is observed at 2 and 3 hours as a light blue shade. After 4 hours a strong blue shade is observed, indicating the release of the liquid that was stored within the implant. At last, with the tests that were performed up until this moment the static emptying time was approximately 6 hours (see FIG. 8 and table 2).

It is worth noting that after this time period had elapsed, even after a 1-week limit, if the system is moved the implant is still able to release solution.

TABLE 2
Observed results for the prolonged fluid
release test from the implant.

| Time elapsed since immersion | Observed color or shade |
|---|---|
| of the implant (hours) | description |
| 1 | Almost transparent liquid, |
| | slight light blue coloration |
| 2 | Light blue shade |
| 3 | Light blue shade |
| 4 | Strong blue shade |
| 6 | Blue shade |

### Example 3: Testing of the Emptying Time Verification of the Implant with One Reservoir

Multiple tests were carried out to verify the emptying time of the 5 mL capsule that is stored within the one-reservoir prototype implant. Furthermore, static and in-movement tests were performed.

For the analyses, an implant was standardized and produced with the following technical characteristics:
2.2 mm diameter plug
1 mm insert diameter (syringe insertion diameter)
Funnel system to reduce liquid overflow at the implant inlet.
5 mL capsule
Walls of a thickness of 2.5 mm at least in every capsule or deposit area
1.4 mm exit
Funnel shaped exit to increase emptying time of the capsule or deposit

Static Assay

On its first stage, the experiment consisted on filling the capsule or reservoir with a solution of water-diluted methylene blue in a 5:1 ratio, this liquid being inserted into the implant and verifying that the total capacity of the chamber or reservoir is properly filled to its total extent.

Then, a plastic container is filled with water, the implant with liquid inside is put on its interior and is anchored with metallic hooks that were set in the plastic container so it can keep a vertical position, mimicking how this implant would be positioned in reality.

The following time periods were assessed: 24H after the start of the experiment (first sample), 72h after the start of the experiment (second sample), 96h after the start of the experiment (third sample), 120h after the start of the experiment (fourth sample), 144h after the start of the experiment (fifth sample), 168h after the start of the experiment (sixth sample).

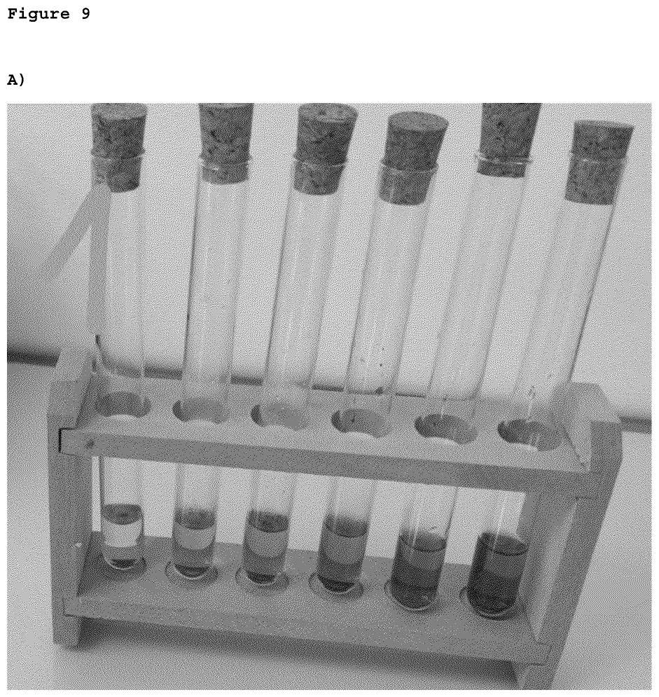

Results for the observation of the retrieved samples for each timepoint allow to indicate at a glance an evident increase in the concentration of the liquid that was released in a prolonged manner to the environment, in this case the container (FIGS. 9A and 9B).

Results for the observation of the samples obtained at each timepoint are described in table 3. At 24 hours little coloration of the environment or liquid release from the implant is observed. At the intermediate timeframes, that is, 72, 96 and 120 hours, an increase release of the liquid is observed, showing a light blue shade coloration of the environment (container) and then at 144 hours the shade changes to a strong light blue shade. At the 168 hour evaluation a greater release of the contents inside the reservoir or capsule of the implant into the environment (container). It should be noted that at day 11 there is still a weak dripping from the implant exit, that still changes the methylene blue concentration in the water of the plastic container.

TABLE 3
Results for the testing of the emptying
time of the implant with one reservoir.

| Time elapsed since | |
|---|---|
| immersion of the | |
| implant (hours) | Observed color or shade description |
| 24 | Almost transparent liquid, slight light |
| | blue coloration |
| 72 | Light blue shade |
| 96 | Light blue shade |
| 120 | Light blue shade |
| 144 | Strong blue shade |
| 168 | Very noticeable strong blue shade |

Dynamic Testing (Moving Implant)

After the tests were performed on static conditions, a new test was performed whose objective is to recreate on a more precise manner how is that this implant-like medical device will behave when it is used by a patient. In that sense, movements that are of typical occurrence on a person (head turning, getting up, walking) were recreated. For this, a 3D machine that has the capacity to horizontally move its platform was used. The water-filled container with the previously described methylene blue solution-filled implant (example 2) inside was placed on top of this machine. The implant type that was used at the testing also corresponds with an implant whose characteristics were described in the application example 2.

Characteristics of the Experiment:
Movement distance: 23 centimeters
Movement speed: 3.3 (cm/s) or 0.1188 (km/h)
A cycle corresponds to the movement from the starting position to the final position (23 centimeters) and going back to the starting position, 46 cm of distance travelled.
Cycles per minute: 3.
Functioning period: 48 h.

After 48 h had elapsed the test was stopped. After this time period had elapsed, methylene blue concentration was observed to be higher at 48 hours in comparison with what was observed in the environment for 72 hour timepoint on the static test (example 2).

### Example 4: Craniofacial Implant to Avoid Post-Implant Infections

After the proposed iterations in application example 1, better implant conditions, sizes and dimensions were determined to allow for a proper and gradual release of a liquid of interest (antibiotic and/or analgesic).

A craniofacial implant produced from polyether ether ketone (PEEK) material is provided to avoid and reduce postoperative infections at the implant site, because said implant comprises:

One or more internal ovoid chambers or reservoirs (1) with walls 2.5 to 4 mm thick, that store a liquid or active ingredients in liquid form to be released in a gradual and prolonged manner into the implant site,
tubes (2) that interconnect the internal chambers or reservoirs with the entry and exit openings, defined as:
   a) an entry tube that connects the entry opening (3) with the reservoir (1) in such a way that it includes a funnel system that includes an 1.5-2.0 mm diameter liquid entry or input that widens to a 2.0-3.0 mm wide tube that then continues to the reservoir.
   b) an exit tube that connects the chamber or reservoir (1) with the exit opening (4) that includes a transition funnel system that is 0.7 to 2 mm wide on its upper part and 0.7 to 1.5 mm wide on its lower part, depending on the diameter of the exit opening (4),

One or more entry openings (3) on the upper part of the implant depending on the number of reservoirs, where said opening is the entry or input for the liquid to reach the reservoir or reservoirs (1), where the opening is 1.5-2.0 mm wide and is blocked by a PEEK filament plug or a titanium screw,

One or more exit openings (4) on the lower part of the implant depending on the number of reservoirs as gradual and prolonged release of the liquid or active ingredient to the implant site, where the exit opening is 0.7-1.5 mm wide and has a plug or system to seal the implant to prevent the escape of the liquid when the implant has not been properly set yet.

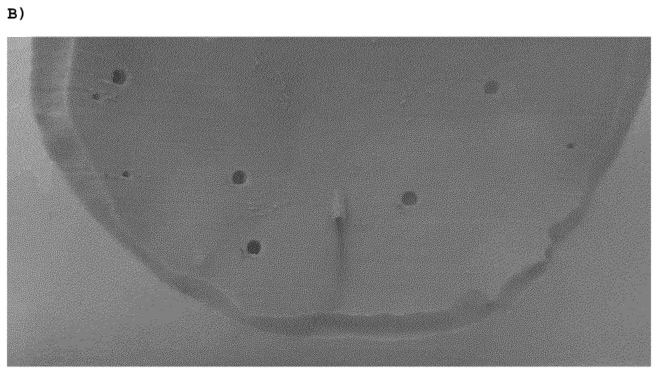

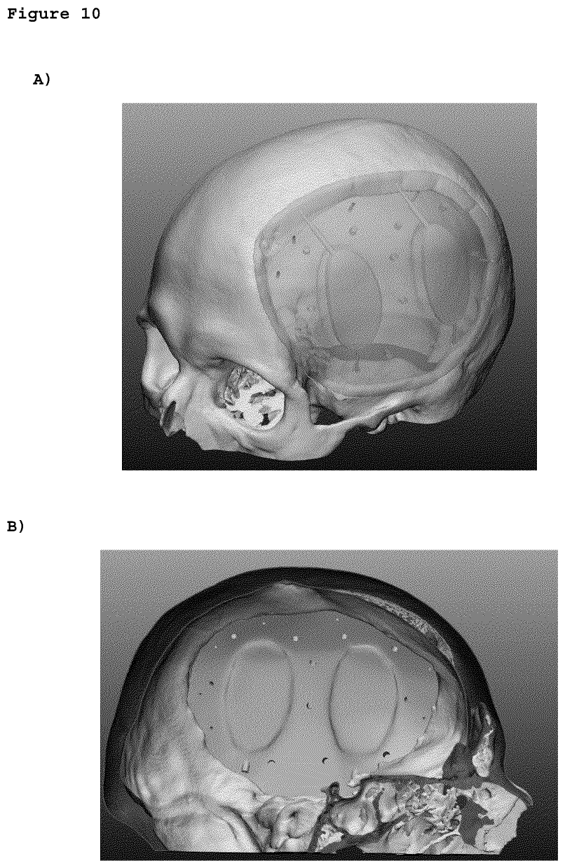

The implant integrated into the skull of the patient then couples and releases the liquid contained within the chambers or reservoirs in a sustained manner thanks to the effect of gravity and the design of the implant. The exit openings release the liquid from the reservoirs to the brain (FIG. 10A ans 10B).

### Example 5: Analytical Study of Vancomycin Release from the Craniofacial Implant

In this study, the release of vancomycin solution from the reservoir of a craniofacial implant part of this invention was reviewed and evaluated. This assay was performed on an incubator set to 37° C. and that was able to perform 20 rpm shaking motion, this was with the intention of mimicking the movement that patients exhibit after this kind of surgery.

To detect the release rate and concentration of the vancomycin released from the implant, a High-Performance Liquid Chromatography (HPLC) coupled to a PDA detector was used. As a standard, vancomycin hydrochloride with a nominal concentration of 0.01 mg/mL in physiological saline solution was used. A calibration curve was generated along with the retention times and area values for each peak present in the chromatogram to determine the vancomycin concentration v/s the standard. For the sampling for released solution from the implant as such, a sample of 0.5 mL was collected and then diluted in mL physiological saline solution to then transfer to a 100 mL flask with 0.2 M potassium dibasic phosphate buffer adjusted to a pH of 7.4. This was then injected in the instrument for analysis.

Vancomycin concentration on the analysis aliquot was determined using the retention times and area values, and then total accumulated vancomycin amount in the vessel was calculated (μg in vessel). Measurements were performed at 1, 2, 4 and 8 hours, the results are presented in table 4.

TABLE 4
Detected area and vancomycin amount
according to sampling times.

| Time (hours) | Area | Sampling volume | μg in vessel |
|---|---|---|---|
| 1 | 263472 | 20 | 13,087.5 |
| 2 | 263981 | 20 | 13,636.3 |
| 4 | 278229 | 40 | 14,868.6 |
| 8 | 326541 | 80 | 18,374.1 |
| 12 | 361050 | 80 | 22,683.5 |

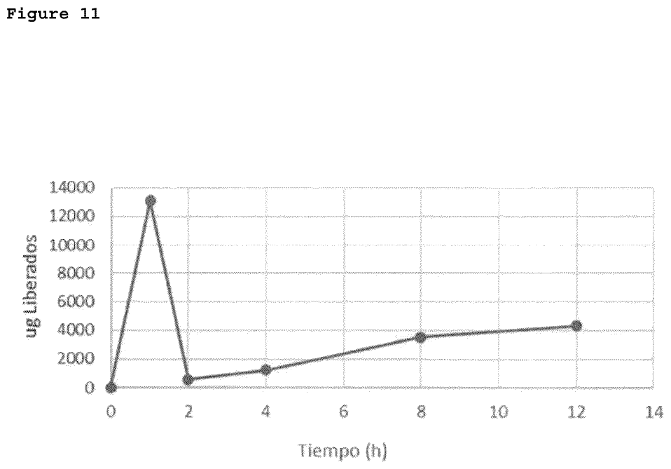

With this sampling and initial calibration an analysis of the amount of released vancomycin per time frame was performed. In table 5 the amount of released vancomycin from the implant (μg) at the evaluated time periods is presented. In FIG. 11, a representative graph for this kinetic release is presented. In accordance with the results, higher vancomycin release from the implant is observed within the first hour frame. From hour 2 and forward, a steady and sustained release is observed in time. This release kinetic allows for an initial exposure of higher active substance amounts in the zone in order to rapidly reach therapeutic concentrations and then a regulated gradual active substance release in time.

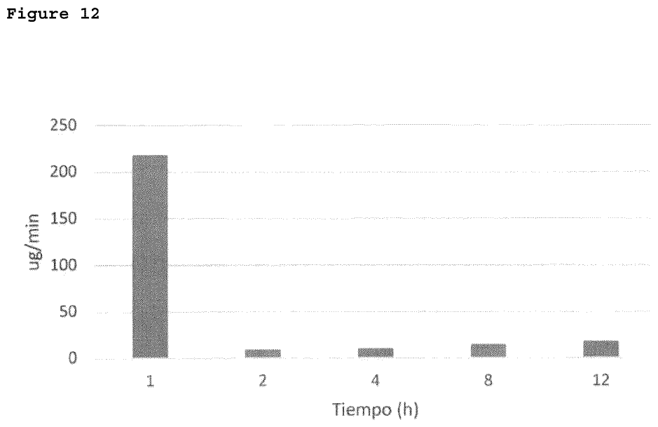

Regarding the release rate at the first hour a high vancomycin release rate was observed with a mean value of 218 μg/mL, that then decreased in the time elapsed from the first to the second hour, to then increase and maintain the prolonged release. In FIG. 12 a representative graph is shown for the vancomycin release rate from the implant at the sampling periods mentioned in table 5.

TABLE 5
Released vancomycin amount and release rate.

| | μg released per | Release rate |
|---|---|---|
| Hour | time frame | (μg/min) |
| 1 | 13,087.5 | 218.1 |
| 2 | 548.5 | 9.1 |
| 4 | 1,232.5 | 10.3 |
| 8 | 3,505.5 | 14.6 |
| 12 | 4,309.4 | 18.0 |

The linear equation or function that allows to determine how long the implant will be releasing active substance was determined from samplings of the 2, 4, 8 and 12 hour periods: f(x)=911.6x+11.465.1. With this information the amount of released vancomycin at later time periods was extrapolated and theoretically determined. In table 6 accumulated released vancomycin amounts defined for the 24, 36 and 43 hour time frames are presented. This simulation does not consider that after the 12-hour timepoint, the rate could decrease, and thus increase delivery times.

TABLE 6
Accumulated released vancomycin amounts
determined up to 43 hours.

| Hour (hr) | Released μg in vessel |
|---|---|
| 1 | 13,087.5 |
| 2 | 13,636.3 |
| 4 | 14,868.6 |
| 8 | 18,374.1 |
| 12 | 22,683.5 |
| 24 | 33,343.9 |
| 36 | 44,283.3 |
| 43 | 50,664.6 |

### Example 7: Stability of the Active Substance Solution within the Implant

For this analysis vancomycin was again considered as the active substance to work with, not limiting said analysis to other active substances.

This assay intends to show that vancomycin is set within the implant and does not degrade while it is contained within, therefore allowing for a functional release. For this, a time zero (To) sample corresponding to a sample that was immediately analyzed after its reconstitution was analyzed, along with a sample that was left to rest for 24 hours within the implant. In this way, results from the 24 hour time frame were compared with those of time zero as sample areas and degradation percentage at 24 hours.

Preparation of the sample solution at time zero (To) consisted on taking an ampule of 500 mg vancomycin and adding 5 mL of physiological saline solution. Shaking up to the complete solution of the lyophilized powder, taking 0.5 mL of said ampule, and diluted with physiological saline solution up to 5 mL of final volume. This 5 mL of solution is injected in the reservoir of the implant. In the case of the sample at 24 hours, 0.5 mL of a solution from a 500 mg vancomycin ampule reconstituted in 5 mL of physiological saline solution were taken. Said 0.5 mL were diluted up to 5 mL of final volume using 0.2M potassium dibasic phosphate buffer adjusted to pH 7.4. Then, using a syringe, said volume was loaded in the reservoir or capsule of the implant, the implant was then sealed and incorporated in an empty beaker. Nominal concentration of the solution within the reservoir is mg/mL.

After 24 hours, the chamber or reservoir was opened to extract the 0.5 mL of the solution contained within. Of said 0.5 mL extracted from the reservoir, a 0.1 mL aliquot is taken that then is transferred to a 100 mL flask for its analysis using HPLC, considering the same calibration and standard curves that were described for the previous example.

In table 7 the results for this assay are presented. It is observed that at the 24 hours degradation of vancomycin is not significant.

This results show that the active substance contained within the reservoir or reservoirs of the implant presented in this invention is kept stable for its release.

TABLE 7
Results for the vancomycin contained in the implant
of the present invention stability assays.

| | | | % degradation |
|---|---|---|---|
| | Time 0 | 24 hour period | at 24 hours |
| Sample 1 | 948050 | 94679 | 99.8 |
| Sample 2 | 948671 | 94744 | 99.9 |
| Mean | 948360.5 | 94711.5 | 99.85 |

### BIBLIOGRAPHY

(Kwarcinski, Jeremy & Boughton, Philip & Ruys, Andrew & Doolan, Alessandra & van Gelder, James. (2017). Cranioplasty and Craniofacial Reconstruction: A Review of Implant Material, Manufacturing Method and Infection Risk. Applied Sciences. 7. 276. 10.3390/app7030276).
Chen Y, Zhang L, Qin T, Wang Z, Li Y, Gu B. (2019). Evaluation of neurosurgical implant infection rates and associated pathogens: evidence from 1118 postoperative infections. Neurosurg Focus. 47(2):E6. doi: 10.3171/2019.5.FOCUS18582.
Chengzhe Gao, Zongliang Wang, Zixue Jiao, Zhenxu Wu, Min Guo, Yu Wang, Jianguo Liu, Peibiao Zhang. (2021). Enhancing antibacterial capability and osseointegration of polyetheretherketone (PEEK) implants by dual-functional surface modification, Materials & Design, Volume 205, 109733. https://doi.org/10.1016/j.matdes.2021.109733
Delaney, LJ, MacDonald, D., Leung, J., Fitzgerald, K., Sevit, A M, Eisenbrey, JR, Patel, N., Forsberg, F., Kepler, CK, Fang, T., Kurtz, SM, & Hickok, Nueva Jersey (2019). Liberación de antibióticos activada por ultrasonido de los clips de PEEK para prevenir la infeccion por fusión espinal: evaluaciones iniciales. Acta biomaterialia, 93, 12-24. https://doi.org/10.1016/j.actbio.2019.02.041
Gu Xinming, Sun Xiaolin, Sun Yue, Wang Jia, Liu Yiping, Yu Kaixuan, Wang Yao, Zhou Yanmin. (2021). Bioinspired Modifications of PEEK Implants for Bone Tissue Engineering. Frontiers in Bioengineering and Biotechnology, Vol. 8, doi:10.3389/fbioe.2020.631616.
Hernandez Cantu, Enoc Isai; Esparza Davila, Sandra Paloma; Reyes Silva, Alan Karim Sayeg. (2020). Eficacia de un modelo de prevencion de infeccion de sitio guirurgico en un hospital de segundo nivel de atención. Index Enferm, Granada, v. 29, n. 1-2, p. 9-12.
Lazar, M. A., Vodnar, D., Prodan, D., Rotaru, H., Roman, C. R., Sorcoi, L. A., Baciut, G.; Campian, R. S. (2016). Antibacterial coating on biocomposites for cranio-facial reconstruction.Clujul medical (1957), 89(3), 430-434. https://doi.org/10.15386/cjmed-599
MINSAL. Ministerio de Salud de Chile. Departamento de Calidad y Seguridad en la atencion, Programa de control de IAAS. (2017). Informe de vigilancia de infecciones asociadas a la atencion de salud. https://www.minsal.cl/wp-content/uploads/2015/09/informe-vigilancia-2017.pdf
Xue, Z., Wang, Z., Sun, A., Huang, J., Wu, W., Chen, M., et al. (2020). Rapid construction of polyetheretherketone (PEEK) biological implants incorporated with brushite (CaHPO4·2H2O) and antibiotics for anti-infection and enhanced osseointegration. Mater. Sci. Eng. C Mater. Biol. Appl. 111:110782. doi: 10.1016/j.msec.2020.110782
Yan, J., Zhou, W., Jia, Z., Xiong, P., Li, Y., Wang, P., et al. (2018). Endowing polyetheretherketone with synergistic bactericidal effects and improved osteogenic ability. Acta Biomater. 79, 216-229. doi:

1. A craniofacial implant composed of PEEK material for the release of substances or active ingredients CHARACTERIZED because it comprises:
One or more internal ovoid chambers or reservoirs (1) with walls 2.5 to 4 mm thick, that store a liquid or active ingredients in liquid form to be released in a gradual and prolonged manner into the implant site,
tubes (2) that interconnect the internal chambers or reservoirs with the entry and exit openings, defined as:
a) an entry tube that connects the entry opening (3) with the reservoir (1) in such a way that it includes a funnel system that includes an 1.5-2.0 mm diameter liquid entry or input that widens to a 2.0-3.0 mm wide tube that then continues to the reservoir.
b) an exit tube that connects the chamber or reservoir (1) with the exit opening (4) that includes a transition funnel system that is 0.7 to 2.0 mm wide on its upper part and 0.7 to 1.5 mm wide on its lower part, depending on the diameter of the exit opening (4),
One or more entry openings (3) on the upper part of the implant depending on the number of reservoirs, where said opening is the entry or input for the liquid to reach the reservoir or reservoirs (1), where the opening is 1.5-2.0 mm wide and is blocked by a PEEK filament plug or a titanium screw,
One or more exit openings (4) on the lower part of the implant depending on the number of reservoirs as gradual and prolonged release of the liquid or active ingredient to the implant site, where the exit opening is 0.7-1.5 mm wide and has a plug or system to seal the implant to prevent the escape of the liquid when the implant has not been properly set yet.

2. A craniofacial implant composed of PEEK material for the release of substances or active ingredients as in claim 1 CHARACTERIZED because the entry opening has a 1.5 mm diameter.

3. A craniofacial implant composed of PEEK material for the release of substances or active ingredients as in claim 1 CHARACTERIZED because the exit opening for the liquid or active ingredient has a mm diameter.

4. A craniofacial implant composed of PEEK material for the release of substances or active ingredients as in claim 1 CHARACTERIZED because the thickness of the chambers or reservoirs is 3.5 mm.

5. A craniofacial implant composed of PEEK material for the release of substances or active ingredients as in claim 1 CHARACTERIZED because the tubings of the whole system, both entry and exit systems, must be at least 10 mm and up to of 15 mm long.

6. A craniofacial implant composed of PEEK material for the release of substances or active ingredients as in claims 1 to 5 CHARACTERIZED because it allows for the prolonged and sustained release of substances or active ingredients in liquid form.

7. Use of the craniofacial implant composed of PEEK material for the release of substances or active ingredients as in accordance with claim 6 CHARACTERIZED because it allows for the prolonged and sustained release of at least one active ingredient soluble in a pharmaceutically accepted vehicle.

8. Use of the craniofacial implant composed of PEEK material for the release of substances or active ingredients in accordance with claim 7 CHARACTERIZED because it allows for the prolonged and sustained release of analgesic, antibiotic, antiviral, chemotherapeutic, anti-inflammatory or any substance or active ingredient soluble in a pharmaceutically acceptable vehicle.
